(19) United States
(12) Patent Application Publication
(10) Pub. No.: US 2002/0103515 A1
(43) Pub. Date: Aug. 1, 2002

(54) Magnetic fields for the treatment of cancer and to assist in nerve regeneration

(76) Inventors: Kent Davey, New Smyrna Beach, FL (US); Truman Veran Williams III, Duluth, GA (US)

Correspondence Address: Levisohn, Lerner, Berger & Langsam, Suite 2500, 757 Third Avenue, New York, NY, 10017, US

(21) Appl. No.: 10/011,792

(22) Filed: Dec. 4, 2001

Related U.S. Application Data
(60) Provisional application No. 60/250,753, filed on Dec. 1, 2000.

(51) Int. Cl. A61N 1/08

(52) U.S. Cl. 607/66

(57) ABSTRACT

A device for cancer treatment and nerve regeneration that induces a non-symmetrical electric field. This electric field is thought to cause electro-osmotic activity around p53 proteins within the body, in addition to altering the transmembrane potential. Both closed and open magnetic field structures can be used towards this end. With a closed magnetic structure, the field is contained within a toroid or similar structure, and the electrical circuit is completed by placing the toroid in a conducting liquid or gel. Using an open circuit, a cut C core of ferromagnetic material is wound with a figure “8” shaped coil and the field is driven into the body. In both cases, the current driving the coil is preferentially asymmetrical, resulting in an induced E field which is not the same in the first half of the cycle as it is in the second. The induced electric field moves organelles into the nerve tip site through the process of electrokinectics.

### RELATED APPLICATIONS

This is a U.S. non-provisional patent application claiming priority on U.S. provisional patent application No. 60/250,753 filed Dec. 01, 2000.

### INCORPORATION BY REFERENCE

The disclosures of U.S. Pat. No. 6,132,361 issued on Oct. 17, 2000; U.S. Pat. No. 6,086,525 issued on Jul. 11, 2000; and U.S. Pat. No. 5,725,471, issued on Mar. 10, 1998 are hereby fully incorporated herein by reference.

1. Field of the Invention

The present invention relates generally to the use of magnetic fields in the treatment of cancer and for nerve regeneration, and more specifically to the use of time changing magnetic fields to induce electric fields believed useful to promote the apoptosis of cancer cells and for the regeneration of damaged nerves.

2. Background of the Invention

Description of Related Art

Costa and Hofmann (Costa, J. L., and Hofman, G. A., 1987, “Malignancy Treatment”, U.S. Pat. No. 4,665,898) suggested the use of high intensity pulsed magnetic fields to neutralize and selectively destroy malignant cells. Strong static magnetic fields have been shown to slow the growth of human cancer cells in vitro. Exposure to extremely low frequency magnetic fields has also been shown to lead to apoptotic cell death in human leukemia cells. See, Narita, K., Hanakawa K., Kasahara T., Hisamitsu T., Asano K., 1997 “Induction of Apoptic Cell Death in Human Leukemic Cell Line HL-60, By Extremely Low Frequency Electric Magnetic Fields; Analysis of the Possible Mechanisms in Vitro” In Vivo, Vol. 111, No. 4, pp. 329-35. Santi Tofani has discussed the inducement of cancer cell apoptosis using a combination of static and low frequency magnetic fields. Tofani, S., “Application of Electromagnetics in Treating Cancer”, invited paper in Abstract Proceedings of Progress in Electromagnetics Research Symposium, Taipei, Taiwan, Mar. 22-26, 1999. Tofani's main thesis (is that the combination of these two fields interferes with the intra-cellular signaling and somehow induces apoptosis through a modification of the p53 gene expression. Tofani uses a pair of Helmholtz coils to generate his magnetic field. Tofani S., Barone D., Cintorino M., Ferrara A., Ossola P., Rolfo K., Ronchetto F., Tripodi S. A., Arcuri F., Peroglio F., “Tumor Growth Inhibition, Apoptosis and Loss of p53 Expression induced In Vitro and In Vivo byt Magnetic Fields,” Abstract Proceedings of the American Association for Cancer Research, Philadelphia, Pa., Apr. 1-14, 1999.

A frequently observed event in malignant cancer cells is the alteration of the p53 gene and encoded protein (Kessis, Theodore D., et al., 1993. “Human Papillomavirus 16 E6 Expression Disrupts the p53-mediated Cellular Response to DNA Damage,” Proc. Natl. Acad. Sci. (USA). 90: 3988-92). See, also, Sun, Yi., et al., 1993. “Progression Toward Tumor Cell Phenotype is Enhanced By Overexpression of a Mutant p53 Tumor-Suppressor Gene Isolated From Nasopharyngeal Carcinoma”, Proc. Natl. Acad. Sci. (USA). 90:2827-2831. It is thought that the magnetic field can decouple the electron spin from the nuclear spin in free radicals. Once the spins are disassociated from the nucleus, two free radicals can recombine in a singlet state that is not chemically reactive with other reagents. The p53 protein is frequently deficient or found to be mutated in tumor cells. Many researchers are now convinced that the p53 protein is actually a growth inhibitor or suppressor (Malkin, David., 1994. “Germline p53 Mutations And Heritable Cancer”, Annu. Rev. Genetics., 28:443-465 and Baker, J. Frank, “A Review Of The Importance Of The p53 Gene In Cancer Research”, 1996, web cite:http://members.aye.net/˜jfbaker/. Like many polyelectrolytes, a dissociation of ionizable groups on the protein can lead to the presence of a net charge on the structure. In such cases, a charge results on the protein and an opposite charge in the surrounding electrolytic solution.

Al Grodzinsky explored the electrokinectics of electric field interaction with charged proteins, especially collagen. Grodzinsky, A. J., Electromechanics of Deformiable Polyelectrolytic Membranes, Ph.D. Thesis, MIT, June, 1974. The two primary types of interactions are shown in FIG. 1. Inset (a) shows a charged protein fiber. Electric forces pull ions of the opposite sign to the wall of the charged protein. Diffusive forces drive the ions back away from the wall. The sign of the charge can be either positive or negative, and will change depending of the pH of the medium. There the protein is shown charged positively. The negative charge in the surrounding electrolytic solution is mobile. When an electric field is impressed, the fluid will move and create a pressure differential from right to left.

Inset (b) shows a freely suspended positively charged protein. An impressed field will force the mobile negative charge downward and the free body protein upward. The action is similar to rowing on a lake. Electrokinectic activity may be a mechanism by which an induced or impressed electric field can influence a p53 protein. Perhaps a small change in transmembrane potential is all that is needed. Cancerous cells exhibit a smaller transmembrane potential. Regardless of the mechanism, induced electric fields appear to have the ability to cause apoptosis in cancer cells.

The use of magnetic fields for nerve regeneration is also receiving considerable attention. Rosner, et.al contend that the magnetic fields align the collagen gel surrounding the nerve cells. The inventors maintain that this is happening through electrokinectic activity. B. I. Rosner, N. Dubey, P. C. Letourneau, R. T. Tranquillo, “Simulated Peripheral Nerve Regeneration in Magnetically Aligned Collagen Gels Derivatized with Laminin Peptides and Seeded with Schwann Cells”, Proceedings of the First Joint BMES/EMBS Conference, ISBN: 0-7803-5674-8, Vol. 1, October, 1999, p. 117. Much of the latter is explained through the work of Alan Gradzinski at MIT (Elliot H. Frank and Alan J. Gradzinsky, “Cartilage Electromechanics—I. Electrokinectic Transduction and the Effects of Electrolyte pH and Ionic Strength”, J. Biomechanics, Vol. 90, No. 6, 1987, pp. 615-627). Note that it is not the magnetic field per se, but the induced electric field which is able to influence the collagen. The collagen has an interfacial charge resulting in a charged cloud near that interface.

The fact that regeneration may be caused by an electic field is underscored by Sweeney et al. (J. D. Sweeney, K. Mosallaie, “An Electrodiffision Model of Low-intensity Electric Field Effects on Early Myelinated Nerve Regeneration” IEEE 17^{th} Annual Conference in Engineering in Medicine and Biology, Sep. 20-23, 1995, Arizona State Univ., Tempe, Ariz., Vol. 2, ISBN: 0-7803-2475-7, pp. 1499-1500. and Schmidt et al. (C. E. Schmidt, V. R. Shastri, E. J. Furnish, R. Langer, “Electrical Stimulation Of Neurite Outgrowth and Nerve Regeneration”, University of Texas, Proceedings of the 17^{th} Southern Biomedical Engineering Conference, Feb. 6-8, 1998, p. 117). Electrophoresis is the movement of the surrounding electrolytic solution when an E field is impressed. Sweeney maintains that the resulting electrophoresis due to the impressed E field results in increased organelle movement towards the tip of the regeneration plate, what he calls the nerve tip. Schmidt is witnessing neurite outgrowth and nerve regeneration with only electric fields. There is a group at U. Michigan under the auspices of Bradley that has designed a perforated electrode which they place between the cut ends of the glossopharyngeal nerve in rats. (T. Akin, K. Najafi, R. H. Smoke, R. M. Bradley, “A Micromachined Silicon Sieve Electrode for Nerve Regeneration Applications” IEEE Transactions on Biomedical Engineering, Vol. 41, No. 4, 1994,pp. 305-313. They are witnessing the regrowth of the nerve through the perforations.

### SUMMARY OF THE INVENTION

It is an object of this invention to provide a means of inducing an electric field, preferentially asymmetrical, to enhance the apoptosis of malignant cells. Ions have an inertial residence time. A small force applied over a long time will have a different displacement effect on an ion than a large force over a small time. For this reason it is thought that an asymmetrical field is superior to a sinusoidal or other symmetrical field pattern to enhance the electrokinectic activity, hopefully useful in cancer treatment. The present invention seeks to induce the desired electric field using a closed ferromagnetic toroid. The use of a ferromagnetic core has already been suggested in the literature for stimulating nerves (Carbunaru, R. and Durand, D. M., “Toroidal Design for Efficient Transcutaneous Magnetic Stimulation of Nerves,” Annals of Biomedical Engr., 41, (11), pp. 1024-1030, 1998.

In the preferred embodiment of the present invention, a toroid is used which rests flat against the patient. The center and sides of the toroid are filled with a conducting gel to provide a closed path for induced ions. In a secondary embodiment, an open or cut ferromagnetic core in the shape of a “C” or “U” is used to drive magnetic flux into the patient. An asymmetrical current is generated either with a signal generator and amplifier, or with a resonant inductive capacitor circuit. Both the closed and open ferromagnetic cores are fabricated on a bobbin by a 2-4 mil thick rolled laminate. It is believed that this invention encourages free radicals to recombine through disassociating the electron spin from the spin of the nucleus, and then encouraging recombination in a singlet state through electrokinetic activity (electro-osmosis). A secondary effect of the induced electric field is the movement of organelles towards the site of an injured nerve, aiding nerve regeneration.

### BRIEF DESCRIPTION OF THE DRAWINGS

FIG. 1 illustrates Electroosmosis (a) and Electrophoresis (b) charged bodies with a diffuse double layer.

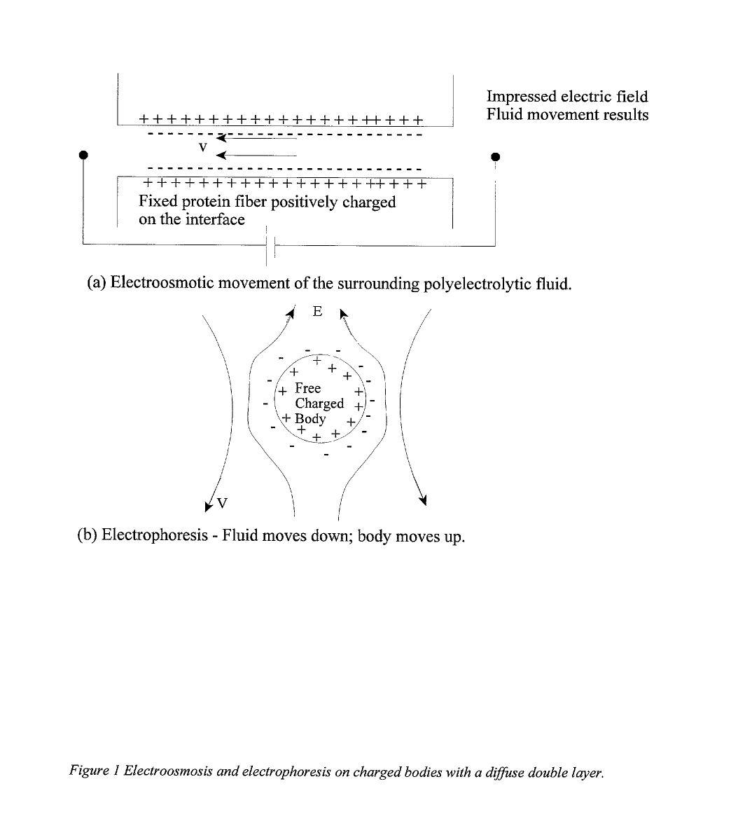

FIG. 2 is a perspective view of a wound toroid made of a ferromagnetic core, placed external to a representative stimulation site for treatment.

FIG. 3 is a perspective, cut-away view of a dome-shaped toroid surrounded and filled by a conducting clay or gel, such as agar.

FIG. 4 is a side view of a patient, showing a cut ferromagnetic core (in a C-shape) and electric coil therearound, used to induce currents within the body for cancer treatment.

FIG. 5(a) is a schematic resonance full circuit useful in obtaining asymmetrical current for use with the present invention; FIG. 5(b) is a simplified equivalent of the circuit.

FIG. 6(a) is a schematic of a negative power supply voltage V providing an alternative semi-resonant full circuit; FIG. 6(b) is a simplified equivalent.

FIG. 7(a) is a schematic of a full circuit with a diode shorting the inductor for accomplishing the role of a voltage source to realize a ramp current wave form as the inductor discharges its energy through the diode; FIG. 7(b) is a simplified equivalent.

### DETAILED DESCRIPTION OF THE INVENTION

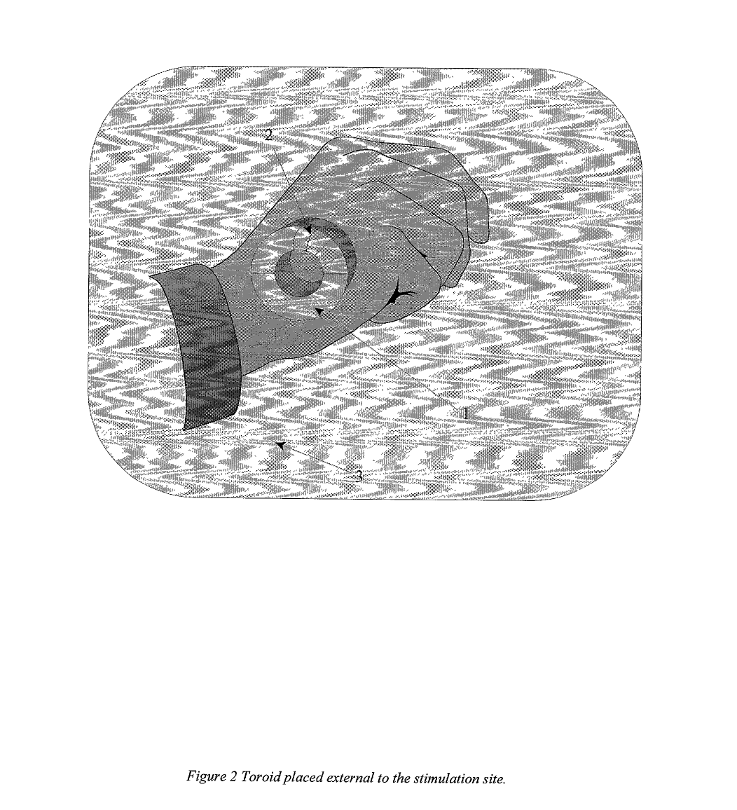

One preferred embodiment of the invention is a simple closed toroid as depicted in FIG. 2. The toroid is wound on a mandrel using a ferromagnetic tape. The wound core may be impregnated with epoxy. A typical tape thickness would be 2-8 mil. The tape is desired to lower eddy currents induced in the core by the changing current excitation.

The toroid 1 is wound with turns 2 to drive an azimuthal flux around the coil. The magnetic flux remains confined within the toroid. If the flux or driving current is changing with time, an electric field is induced through and around the perimeter of the toroid. No electrical current will actually flow unless the secondary electrical circuit is closed. One means by which that is accomplished is by immersing the toroid and the hand in a saline solution 3.

Although the saline allows a current completion path, it is not very convenient. The completion path can also be realized through an agar gel, a thick conducting paste, or even a wet clay. The only believed requirement is that it be reasonably conductive to facilitate ion flow, preferably having a conductivity greater than 0.1 mhos/m.

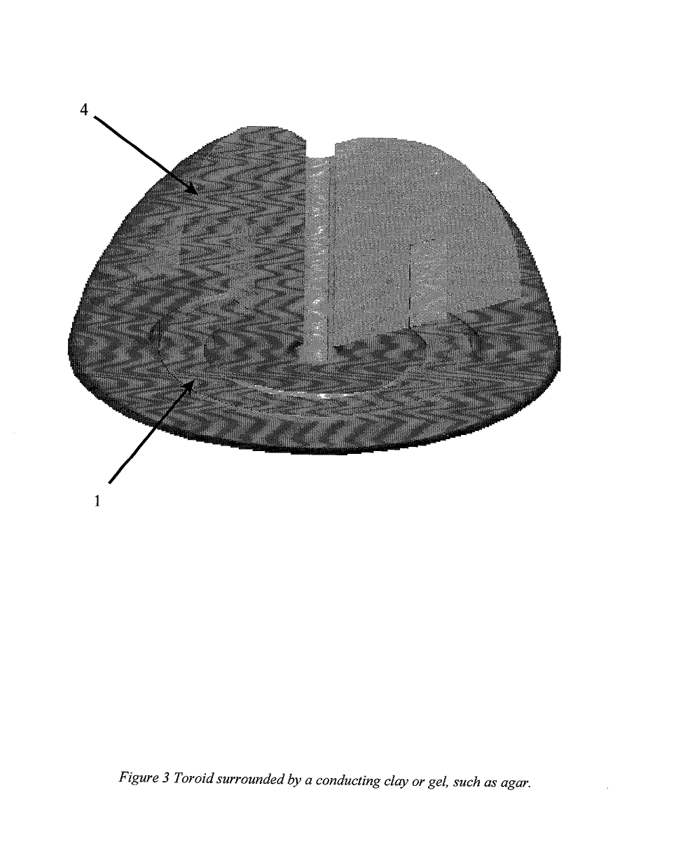

FIG. 3 shows ¾ of a toroid 1 shown surrounded on its upper side by such a clay or agar gel 4. The hole in the center of the mold is unnecessary and can conveniently be filled. The electrical wires exciting the toroid are not shown in this drawing.

The toroid device can be positioned on a supine patient ready for treatment, for example on an upper chest tumor. An electrolytic paste 5 would be applied to the patient's chest to enhance the electrical contact of the gel with the patient. A changing magnetic field will induce a current through the center of the toroid down into the patient's chest, and back up into the gel, completing its path around the toroid.

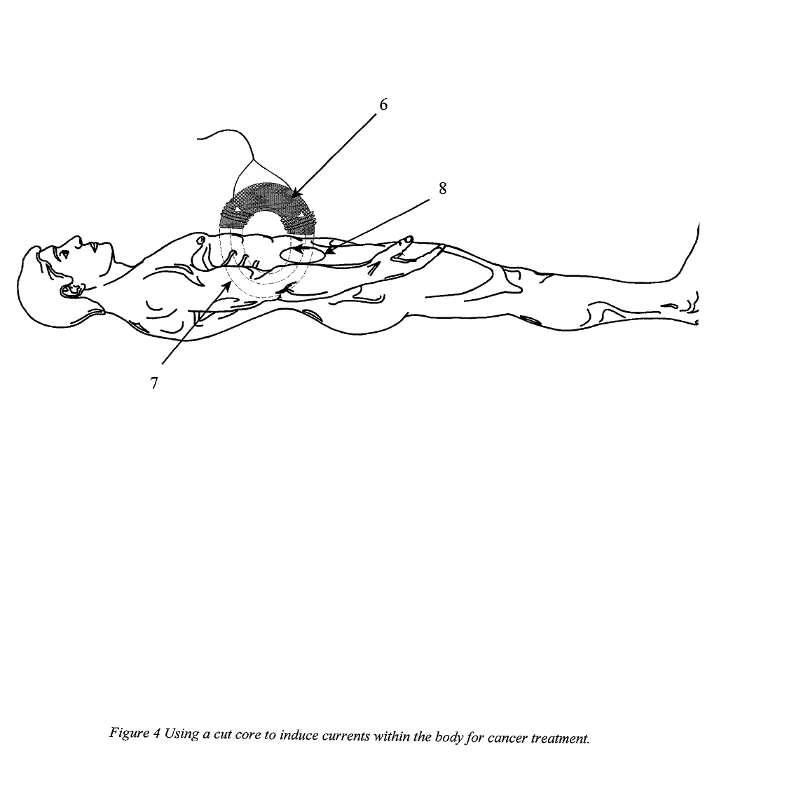

An alternative embodiment which allows treatment through clothing and which might be more suitable for certain regions (e.g., anal or vaginal) is shown in FIG. 4. The toroid 6 is a cut toroid allowing the magnetic field 7 generated by the toroid to penetrate the body and induce currents 8 within the body. The cut C core can be wound with a figure “8” shaped coil. Like the complete toroid 1, this cut toroid 6 is laminated and wound with a tape on a mandrel. The laminations must be epoxy impregnated before cutting. The cut edges must then be deep acid etched to prevent the laminations from shorting to one another at the cut. The cut edge is then brush epoxyed after cutting. The advantage of the cut core is its convenience; no gel or electrolytic paste is required and it works through the clothing. The disadvantage is that the current must be considerably larger due to the large air gap.

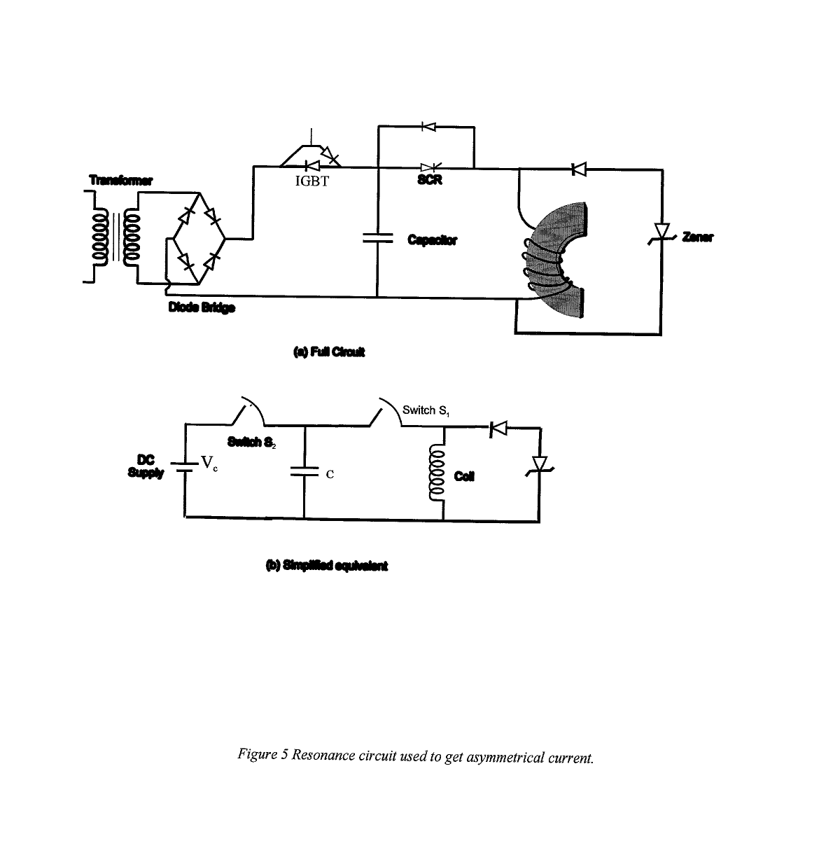

The preferred current excitation for either the closed or cut cores is a ramp of current. This can be achieved through a function generator having this capability followed by a current amplifier. Typically such devices, especially cut cores, are excited using a resonant circuit. The most natural inductive-capacitive (LC) circuits that accomplish this use a full current sine wave cycle. As explained above, the more desirable current waveform is one that is asymmetrical. At least one way to utilize the LC circuit and still get the asymmetrical ramp-like current pulse is shown in FIG. 5. The capacitor is charged using a standard transformer-bridge circuit. The charged capacitor resonates with the inductive core when the thyristor fires. The parallel path zener diode and reverse diode combination ensures that the reverse current through the core will discharge with a different time constant through this alternative path. The sinusoidal current rises for a quarter cycle and then decays with an L/R type time constant through the zener diode path, resulting in a ramp-type waveform. A resistor in series with the zener may be added to help dissipate heat for larger power applications.

The semi-resonant circuit has two advantages over the function generator-amplifier approach. First, the circuit is simpler. Second, the semi-resonant circuit uses less energy. The function generator-generator circuit has to supply the initial energy for the forward pulse of current (½ LI²) plus that necessary to drive the current down to zero through the amplifier circuitry. The semi-resonant circuit supplies only the initial ½ LI² energy.

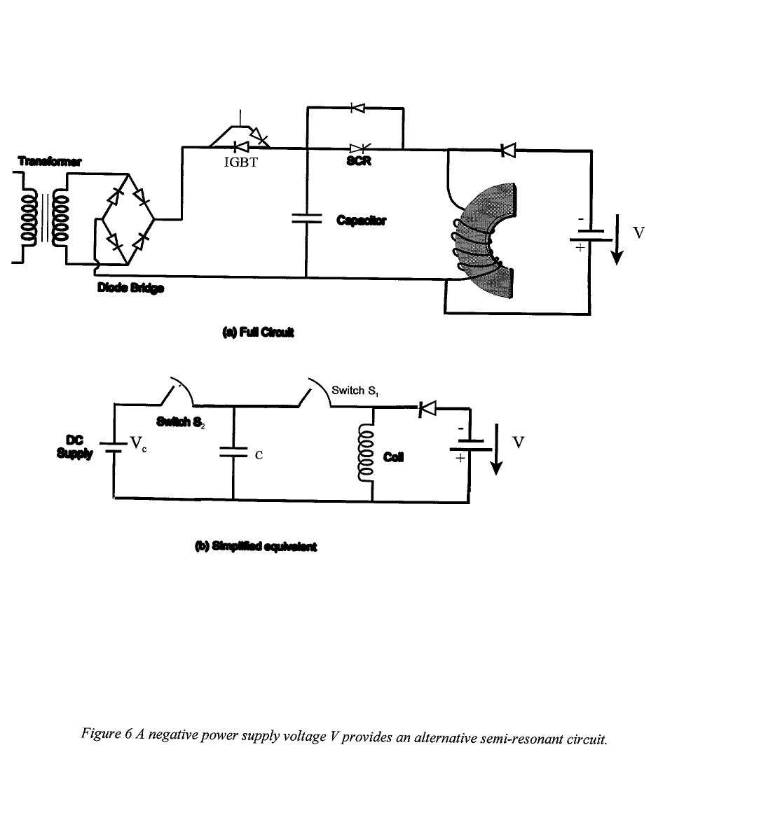

An alternative circuit accomplishing the same end with a bit more flexibility is realized by replacing the zener by a negative voltage supply as suggested in FIG. 6. The size of the negative voltage supply dictates the slope of the decaying current.

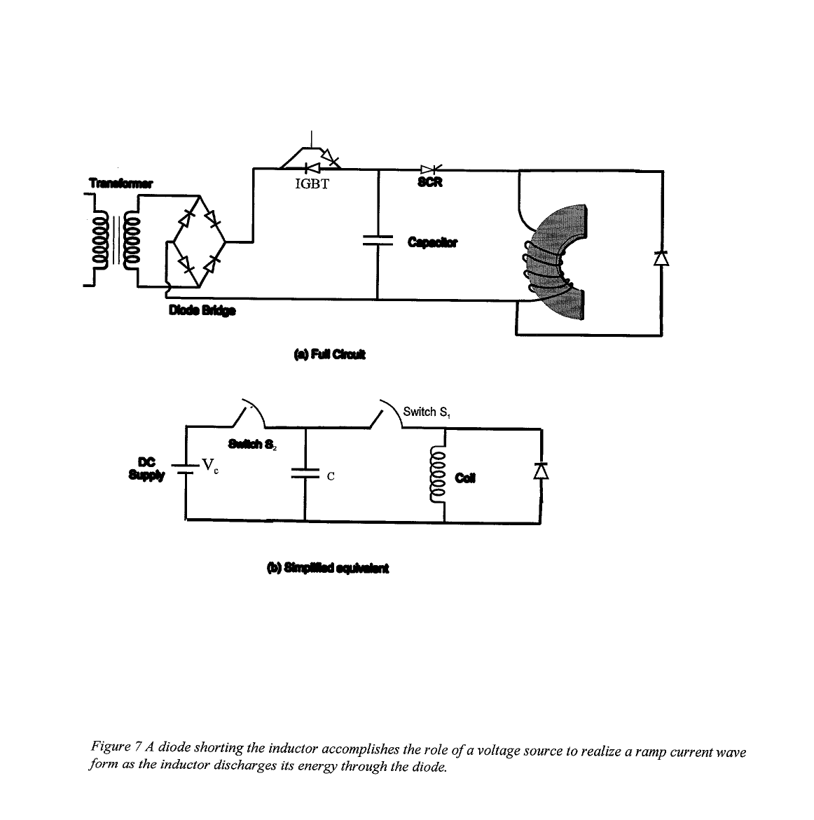

An alternate embodiment for accomplishing the ramp current excitation is shown in FIG. 7. The diode is simply shorting the inductor allowing its current to decay an L di/dt equal to the voltage drop across the diode. Multiple diodes can be employed to slow or speed up the decay of energy from the inductor. Note that in this embodiment, less diodes are required since the diode itself will back bias the thyrister. A diode in parallel with the thyrister, as in FIG. 6, is also unnecessary.

The same circuit is believed to be useful for nerve regeneration. When a cell wall has a net charge associated with it, oppositely charged mobile ions are attracted to the cell. The result is a mobile cloud charge around the cell. When the cell is exposed to an electric field, the mobile charges move in response to the electric field. If the electric field is asymmetrical, a net movement in one direction can be achieved, due to the fact that the mobile ions cannot effectively respond to a rapid momentary reversal. The result is that the electric field can supply nutrients and organelles to the cell interface which appear to be useful in the regeneration process.

Having described this invention with regard to specific embodiments, it is to be understood that the description is not meant as a limitation since further embodiments, modifications and variations may be apparent or may suggest themselves to those skilled in the art. It is intended that the present application cover all such embodiments, modifications and variations.

1. An apparatus for the treatment of cancer comprising:
a) a ferromagnetic toroid core wrapped with a wire coil; and
b) an electric circuit capable of supplying a non-symmetrical electric field to said coil.

2. The apparatus of claim 1 wherein the sides and center of said toroid are filled with a conducting gel or paste.

3. The apparatus of claim 1 wherein said toroid is formed by wrapped around a magnetically saturable material at least one layer of a ferromagnetic tape.

4. The apparatus of claim 1 wherein said toroid is cut open so as not to extend 360.

5. The apparatus of claim 1 wherein said toroid has a flat surface for lying flat against the surface a patient.

6. An apparatus for the stimulation of nerve regeneration comprising:
a) a ferromagnetic toroid core wrapped with a wire coil; and
b) an electric circuit capable of supplying a non-symmetrical electric field to said coil.

7. The apparatus of claim 6 wherein the sides and center of said toroid are filled with a conducting gel or paste.

3-1. The apparatus of claim 6 wherein said toroid is formed by wrapped around a magnetically saturable material at least one layer of a ferromagnetic tape.

4-1. The apparatus of claim 6 wherein said toroid is cut open so as not to extend 360.

5-1. The apparatus of claim 6 wherein said toroid has a flat surface for lying flat against the surface a patient.

11. A method for the detecting cancer wherein a ferromagnetic toroid may excite both a closed and open core using an LC circuit and a zener diode to produce an asymmetrical current excitation ramp waveform.

12. The method of claim 11, wherein said ferromagnetic toroid may excite both a closed and open core using a LC circuit and a negative voltage source to produce an asymmetrical current excitation ramp waveform.

13. The apparatus of claim 11, wherein said ferromagnetic toroid may excite both a closed and open core using a LC circuit and a shorting diode across the inductor to produce an asymmetrical current excitation ramp waveform.

14. A method for the stimulation of nerve regeneration wherein a ferromagnetic toroid may excite both a closed and open core using an LC circuit and a zener diode to produce an asymmetrical current excitation ramp waveform.

15. The method of claim l4, wherein said ferromagnetic toroid may excite both a closed and open core using a LC circuit and a negative voltage source to produce an asymmetrical current excitation ramp waveform.

16. The method of claim 14, wherein said ferromagnetic toroid may excite both a closed and open core using a LC circuit and a shorting diode across the inductor to produce an asymmetrical current excitation ramp waveform.

17. The method of claim 14, wherein said asymmetrical electrical fields produced causes ion/organelle flow in association with a damaged nerve and results in healing of the nerve.
